(19) United States
(12) Patent Application Publication
(10) Pub. No.: US 2024/0282414 A1
(43) Pub. Date: Aug. 22, 2024

(54) Biological Measurement Data Processing Device and Biological Measurement Data Processing Method

(71) Applicant: Hitachi, Ltd., Tokyo (JP)

(72) Inventor: Shunsuke MINUSA, Tokyo (JP)

(21) Appl. No.: 18/413,818

(22) Filed: Jan. 16, 2024

(30) Foreign Application Priority Data
Feb. 22, 2023 (JP) 2023-025829

(51) Int. Cl. G16H 10/40; A61B 5/00; A61B 5/16

(52) U.S. Cl. CPC G16H 10/40; A61B 5/16; A61B 5/7282; A61B 5/742

(57) ABSTRACT

Provided is a processing device capable of notifying a user of detection of an event at a timing appropriate for the user when the event is detected based on biological measurement data of the user. A biological measurement data processing device according to the invention includes a reception unit configured to receive biological measurement data of a user and situation expression data expressing a situation of the user, an event score evaluation unit configured to evaluate, based on the biological measurement data, an event score indicating a degree to which confirmation or an intervention behavior is to be performed by the user regarding an event that occurred with the user, a situation score evaluation unit configured to evaluate, based on the situation expression data, a situation score indicating a degree of a situation in which the user easily performs a behavior regarding a notification, and a notification score evaluation unit configured to calculate, based on the event score and the situation score, a notification score expressing a degree of appropriateness to notify the user of occurrence of the event, and output the notification score.

### CLAIM OF PRIORITY

The present application claims priority from Japanese Patent Application JP 2023-025829 filed on Feb. 22, 2023, the content of which is hereby incorporated by reference into this application.

### BACKGROUND OF THE INVENTION

### 1. Field of the Invention

The present invention relates to a biological measurement data processing device and a biological measurement data processing method for processing data of a biological condition measured from a user.

### 2. Description of Related Art

A technique of estimating a mental and physical health condition such as an emotion, fatigue, and a mood of a user based on data obtained by measuring a biological condition (biological measurement data) in daily life is being studied. Such a technique can be used to determine whether an abnormality occurs in a living body of a user based on the biological measurement data, and take an intervention measure such as solving the abnormality based on a determination result.

PTL 1 discloses an example of a technique in the related art for determining whether an abnormality occurs in a living body of a user based on biological measurement data.

### CITATION LIST

### Patent Literature

PTL 1: JP2016-122434A

### SUMMARY OF THE INVENTION

A home medical support system disclosed in PTL 1 analyzes biological information (pulse, blood pressure, and oxygen saturation) of a support subject, determines whether there is an abnormality in a living body of the support subject based on a value of the biological information, and issues a notification. Such a technique in the related art is suitable for a case where a medical staff always needs to take measures when there is a notification as in a medical support system.

However, in a case of prompting a user to perform an intervention behavior for improving a condition when a specific event (for example, an abnormality in a subjective condition such as perception of stress) that occurred with the user in daily life is detected and the user is notified of the occurrence of the event, the user may not be immediately notified of the occurrence of the event. Further, when the notification is not issued in a situation where the user can confirm the notification and perform the intervention behavior, an effect of promoting the user to introspect notification contents and perform the intervention behavior is poor. There are the above-described problems in the related art, and a technique capable of notifying detection of an event at a timing appropriate for a user is desired.

An object of the invention is to provide a processing device and a processing method capable of notifying a user of detection of an event at a timing appropriate for the user when the event is detected based on biological measurement data of the user.

A biological measurement data processing device according to the invention includes: a reception unit configured to receive biological measurement data of a user and situation expression data expressing a situation of the user; an event score evaluation unit configured to evaluate, based on the biological measurement data, an event score indicating a degree to which confirmation or an intervention behavior is to be performed by the user regarding an event that occurred with the user; a situation score evaluation unit configured to evaluate, based on the situation expression data, a situation score indicating a degree of a situation in which the user easily performs a behavior regarding a notification; and a notification score evaluation unit configured to calculate, based on the event score and the situation score, a notification score expressing a degree of appropriateness to notify the user of occurrence of the event, and output the notification score.

A biological measurement data processing method according to the invention includes: a reception step of receiving biological measurement data of a user and situation expression data expressing a situation of the user; an event score evaluation step of evaluating, based on the biological measurement data, an event score indicating a degree to which confirmation or an intervention behavior is to be performed by the user regarding an event that occurred with the user; a situation score evaluation step of evaluating, based on the situation expression data, a situation score indicating a degree of a situation in which the user easily performs a behavior regarding a notification; and a notification score evaluation step of calculating, based on the event score and the situation score, a notification score expressing a degree of appropriateness to notify the user of occurrence of the event, and outputting the notification score.

According to the invention, it is possible to provide a processing device and a processing method capable of notifying a user of detection of an event at a timing appropriate for the user when the event is detected based on biological measurement data of the user.

### BRIEF DESCRIPTION OF THE DRAWINGS

FIG. 1 is a block diagram showing an example of a main configuration of a biological measurement data processing system and a biological measurement data processing device according to a first embodiment of the invention;

FIG. 2 is a flowchart showing an example of processing in which the biological measurement data processing device receives data from a user terminal;

FIG. 3 is a flowchart showing an example of processing of training an event evaluation model, which is executed by the biological measurement data processing device;

FIG. 4 is a flowchart showing an example of processing of training a situation expression evaluation model, which is executed by the biological measurement data processing device;

FIG. 5 is a flowchart showing an example of processing of training a notification score evaluation model, which is executed by the biological measurement data processing device;

FIG. 6 is a flowchart showing an example of processing of evaluating a notification score based on biological measurement data and situation expression data, which is executed by the biological measurement data processing device;

FIG. 7A is a flowchart showing a detailed example of event score evaluation processing executed by an event score evaluation unit of the biological measurement data processing device;

FIG. 7B is a flowchart showing a detailed example of situation expression evaluation processing executed by a situation score evaluation unit of the biological measurement data processing device;

FIG. 7C is a flowchart showing a detailed example of notification score evaluation processing executed by a notification score evaluation unit of the biological measurement data processing device;

FIG. 7D is a flowchart showing a detailed example of notification determination processing executed by a notification determination unit of the biological measurement data processing device;

FIG. 7E is a flowchart showing an example of processing of receiving a correspondence result according to a notification output, which is executed by the biological measurement data processing device;

FIG. 8A is a diagram showing an example of a notification output screen displayed on a screen of the user terminal by a result display unit, and shows an example of a display screen based on a notification score;

FIG. 8B is a diagram showing an example of a notification output screen displayed on the screen of the user terminal by the result display unit, and shows another example of the display screen based on the notification score;

FIG. 8C is a diagram showing an example of a notification output screen displayed on the screen of the user terminal by the result display unit, and shows an example of a display screen based on the notification score and an event score;

FIG. 9A is a diagram showing an example of a data structure of event score data stored by the biological measurement data processing device;

FIG. 9B is a diagram showing an example of a data structure of situation score data stored by the biological measurement data processing device;

FIG. 9C is a diagram showing an example of a data structure of notification score data stored by the biological measurement data processing device;

FIG. 9D is a diagram showing an example of a data structure of history data stored by the biological measurement data processing device;

FIG. 9E is a diagram showing an example of a data structure of user feature data stored by the biological measurement data processing device; and

FIG. 9F is a diagram showing an example of a data structure of notification setting stored by the biological measurement data processing device.

### DESCRIPTION OF EMBODIMENTS

The invention relates to a biological measurement data processing device and a biological measurement data processing method used for determining, based on a biological condition measured from a user (biological measurement data), a timing or the like at which the user is in a subjective condition different from a usual condition.

In the biological measurement data processing device and the biological measurement data processing method according to the invention, when a target event (for example, a change in a subjective condition) for which confirmation or an intervention behavior is to be performed is detected based on biological measurement data of a user measured in daily life, the user can be notified of detection of the event at a timing when the user more easily performs the confirmation or the intervention behavior.

In the invention, a notification score is calculated and output based on a situation score that is estimated based on situation expression data and indicates appropriateness of a notification timing, in addition to an event score that is estimated based on biological measurement data and indicates a certainty at which a notification is to be issued. In the invention, since the notification score is used, the user can be notified of the occurrence of the target event at a timing in consideration of appropriateness and immediacy. This leads to promotion of a behavior of the user based on the notification, such as improvement of acceptability of an intervention measure based on the notification.

Hereinafter, a biological measurement data processing device and a biological measurement data processing method according to an embodiment of the invention will be described with reference to the drawings. The biological measurement data processing method according to the present embodiment is performed by the biological measurement data processing device according to the present embodiment.

In the following description, a biological condition of a user refers to a health condition of the user (e.g., physical condition and physiological condition), and includes, for example, a heartbeat, an electrodermal activity, and a movement of the user. Biological measurement data is data obtained by measuring a biological condition of a user.

A subjective health condition of a user refers to a physical and mental condition felt by the user, and is, for example, a condition other than an objective condition obtained by performing a measurement with a sensor, such as values of a blood pressure or a pulse. Examples of the subjective health condition include a condition measured using a psychological questionnaire used in a clinical site, such as a Pittsburgh sleep quality index, a condition such as an attention function and a processing speed measured by a psychological experiment, such as a trail making test, and a condition obtained by measuring a level of an emotion or a magnitude of a stress load at a certain time point or during a certain period of time using a Likert scale or a visual analogue scale. In addition, the subjective health condition of a user includes a physical and mental condition of the user obtained by observation and estimation of the user performed by a person other than the user.

An event refers to a change in a subjective condition of a user or an abnormality in a subjective condition of a user. In an embodiment of the invention, a subjective health condition of a user is an estimation target based on biological measurement data (biological information), and a change or an abnormality in the subjective health condition is detected as occurrence of an event, and the user is notified of the occurrence of the event at a timing appropriate for the user.

### First Embodiment

A biological measurement data processing device and a biological measurement data processing method according to a first embodiment of the invention will be described. The biological measurement data processing device according to the present embodiment can be included in, for example, a biological measurement data processing system.

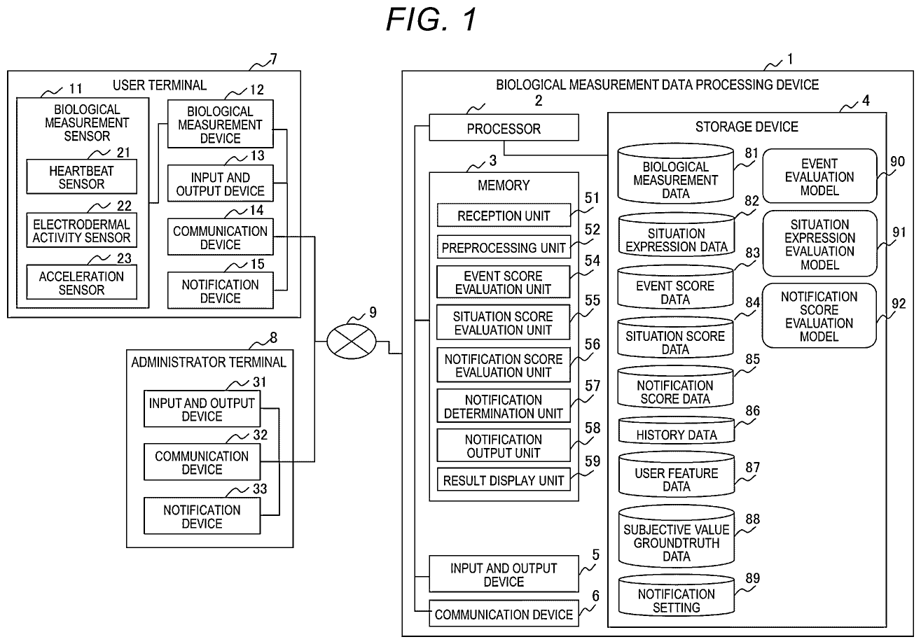

FIG. 1 is a block diagram showing an example of a main configuration of the biological measurement data processing system and the biological measurement data processing device according to the present embodiment. The biological measurement data processing system includes one or more user terminals 7, one or more administrator terminals 8, and a biological measurement data processing device 1 according to the present embodiment. The user terminal 7, the administrator terminal 8, and the biological measurement data processing device 1 are connected to one another via a network 9. The biological measurement data processing device 1 receives data from the user terminal 7 via the network 9 and processes the received data.

The user terminal 7 includes a biological measurement sensor 11 that measures a biological condition of a user, a biological measurement device 12 that controls the biological measurement sensor 11, an input and output device 13, a communication device 14, and a notification device 15, and the user terminal 7 is used by a user. In addition, the user terminal 7 includes a screen and displays a notification to a user, acquired data, and the like on the screen.

The biological measurement sensor 11 includes a heartbeat sensor 21 that detects a heartbeat interval (R-R interval, RRI) of a user, an electrodermal activity sensor 22 that detects an electrodermal activity of a user, and an acceleration sensor 23 that detects a movement of a user. The heartbeat sensor 21 can be, for example, a sensor that detects a heartbeat based on an electrocardiogram, a photoplethysmography, a pressure change, a heart sound, or the like.

The biological measurement sensor 11 is not limited to the above-described sensors, and can include a sensor that detects a body temperature, a blink, an eye movement, myoelectricity, brain activity, which can be measured using electroencephalography, magnetoencephalography, or near-infrared spectroscopy, or the like. The biological measurement sensor 11 can be a wearable device that can be worn by a user, and can be a system incorporated in a smartphone that can be carried by a user.

The biological measurement device 12 controls the biological measurement sensor 11 and generates biological measurement data 81 based on a biological condition measured by the biological measurement sensor 11. The biological measurement data 81 is data measured by the biological measurement sensor 11 and is biological information of a user. The biological measurement device 12 executes calculation processing and reduction processing on a biological condition as needed to generate the biological measurement data 81.

The input and output device 13 displays information on the screen of the user terminal 7, and receives an input from a user. The input from a user includes an input of subjective value groundtruth data 88 which is groundtruth data of a subjective health condition of a user. The groundtruth data of a subjective health condition of a user is a subjective condition of the user recognized by the user, and for example, is input to the user terminal 7 by the user using the input and output device 13.

The communication device 14 executes processing for the user terminal 7 to communicate with the biological measurement data processing device 1 and the administrator terminal 8 via the network 9.

The notification device 15 notifies a user as needed in accordance with an instruction from the biological measurement data processing device 1. For example, the notification device 15 notifies a user of occurrence of an event (a change in a subjective condition of the user or occurrence of an abnormality). The notification device 15 displays a notification on the screen of the user terminal 7 via the input and output device 13. The notification device 15 may notify a user using, for example, a vibration or a sound.

Although the user terminal 7 is implemented by one device in the present embodiment, the user terminal 7 may not necessarily be implemented by one device. For example, the input and output device 13 and the communication device 14 may be provided in a smartphone, the biological measurement device 12, the biological measurement sensor 11, and the notification device 15 may be provided in a smartwatch, and the smart phone and the smartwatch may constitute one user terminal 7.

The administrator terminal 8 includes an input and output device 31, a communication device 32, and a notification device 33, and is used by an administrator. In addition, the administrator terminal 8 includes a screen, and displays a notification to an administrator, acquired data, and the like on the screen.

The input and output device 31 displays information on the screen of the administrator terminal 8 and receives an input from an administrator.

The communication device 32 executes processing for the administrator terminal 8 to communicate with the biological measurement data processing device 1 and the user terminal 7 via the network 9.

The notification device 33 notifies an administrator as needed in accordance with an instruction from the biological measurement data processing device 1. The notification device 33 displays a notification on the screen of the administrator terminal 8 via the input and output device 31. The notification device 33 may notify an administrator using, for example, a vibration or a sound.

The biological measurement data processing device 1 is implemented by a computer, and includes a memory 3, a processor 2, a storage device 4, an input and output device 5, and a communication device 6. The biological measurement data processing device 1 includes, as functional units, a reception unit 51, a preprocessing unit 52, an event score evaluation unit 54, a situation score evaluation unit 55, a notification score evaluation unit 56, a notification determination unit 57, a notification output unit 58, and a result display unit 59. Details of the functional units will be described later.

The memory 3 loads programs for implementing the functional units of the biological measurement data processing device 1. The programs for implementing the functional units are executed by the processor 2.

The processor 2 operates as a functional unit that provides predetermined functions by executing processing according to programs for implementing the functional units. For example, the processor 2 functions as the event score evaluation unit 54 by executing an event score evaluation program. The same applies to other functional units of the biological measurement data processing device 1. The processor 2 also operates as a functional unit that provides functions of a plurality of types of processing executed by programs.

The storage device 4 stores data used by the functional units. For example, the storage device 4 stores the biological measurement data 81, situation expression data 82, event score data 83, situation score data 84, notification score data 85, history data 86, user feature data 87, the subjective value groundtruth data 88, an event evaluation model 90, a situation expression evaluation model 91, a notification score evaluation model 92, and notification setting 89.

The input and output device 5 includes an input device and an output device. Examples of the input device include a mouse, a keyboard, a touch panel, and a microphone. Examples of the output device include a display and a speaker.

The communication device 6 executes processing for the biological measurement data processing device 1 to communicate with the user terminal 7 and the administrator terminal 8 via the network 9.

In the present embodiment, an example will be described in which a user always wears or carries the user terminal 7 in daily life and the biological measurement sensor 11 is constantly in operation. The biological measurement data processing device 1 according to the present embodiment can be used without being limited to an aspect of the example. For example, a user may use the user terminal 7 to operate the biological measurement sensor 11 several times a day such as when the user wakes up or when the user sleeps.

FIG. 2 is a flowchart showing an example of processing in which the biological measurement data processing device 1 receives data from the user terminal 7. The processing is executed by the reception unit 51 of the biological measurement data processing device 1.

When a connection with the user terminal 7 is established via the network 9, the reception unit 51 starts data receiving processing S21 and receives data from the user terminal 7. The data receiving processing S21 is continued until the connection between the reception unit 51 and the user terminal 7 is cut off.

The biological measurement data processing device 1 receives the biological measurement data 81 (biological information) generated by the biological measurement device 12 of the user terminal 7 as data from the user terminal 7. Hereinafter, a case where the biological measurement data 81 is heartbeat interval data, electrodermal activity data, and acceleration data will be described. That is, hereinafter, a case where biological information of a user is a heartbeat interval, an electrodermal activity, and a movement will be described.

When the subjective value groundtruth data 88 is input to the user terminal 7 via the input and output device 13, the biological measurement data processing device 1 receives the subjective value groundtruth data 88. As described above, the subjective value groundtruth data 88 is groundtruth data of a subjective health condition of a user, and the subjective health condition of a user is an estimation target based on the biological measurement data 81 (biological information).

In addition, when a response or the like to a notification to a user is input to the user terminal 7 via the input and output device 13, the biological measurement data processing device 1 also receives, as the history data 86, information of the response or the like to the notification to a user.

Hereinafter, for example, a case where a subjective health condition (an estimation target based on the biological information and a target for which a change or an abnormality is detected as an event) is set as a target of a natural emotion in daily life will be described. In this case, the subjective value groundtruth data 88 can be used to measure a natural emotion in terms of a emotion dimension including an arousal level Arousal and an affective value Valence. When the natural emotion is measured in an emotion dimension, the subjective health condition can be measured using an affective slider that measures the emotion dimension by representing the emotion dimension with picture characters at both ends of a visual analogue scale (VAS) which is a visual scale, or a self-assessment manikin (SAM) that measures the emotion dimension with multi-stage picture characters, and can be represented by a numerical value as a subjective value. The subjective value groundtruth data 88 can be groundtruth data of the subjective value represented by such a numerical value.

Further, the subjective value groundtruth data 88 can be measured in terms of an experience emotion in addition to the emotion dimension. For example, the subjective value groundtruth data 88 can be measured or represented using a positive and negative affect schedule (PANAS) that measures discrete emotions such as pleasure based on a degree of fit to an adjective indicating a emotion.

An example in which the biological measurement data processing device 1 continuously receives the biological measurement data 81 will be described in the present embodiment. The biological measurement data processing device 1 may not necessarily receive the biological measurement data 81 continuously. For example, the biological measurement data processing device 1 may receive the biological measurement data 81 from the user terminal 7 at a regular time interval. The user terminal 7 can collectively transmit the biological measurement data 81 in a plurality of measurement times to the biological measurement data processing device 1 at a regular time interval such as 2 minutes or 30 minutes. The reception unit 51 may establish a connection with the user terminal 7 and execute the data receiving processing S21 only when transmission processing from the user terminal 7 is executed.

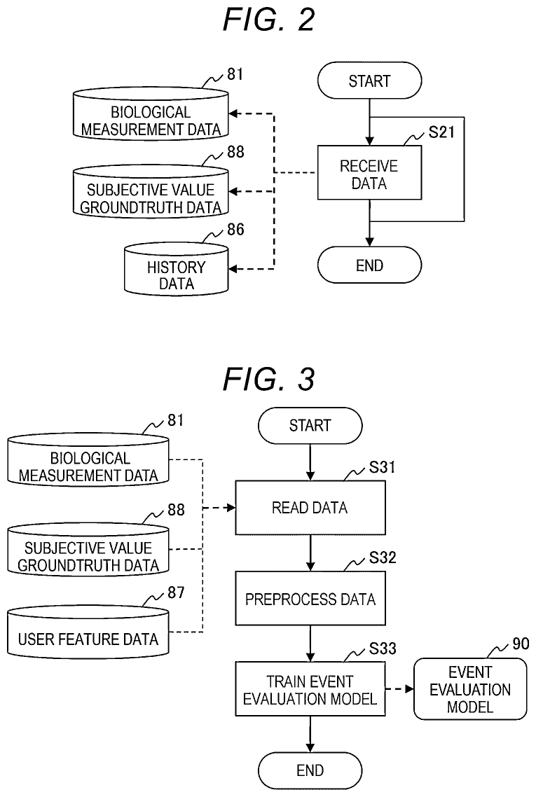

FIG. 3 is a flowchart showing an example of processing of training the event evaluation model 90, which is executed by the biological measurement data processing device 1. The event evaluation model 90 is a model for detecting an event based on the biological measurement data 81.

In data reading processing S31, the reception unit 51 of the biological measurement data processing device 1 receives the subjective value groundtruth data 88, the biological measurement data 81, and the user feature data 87 that are used for training.

The user feature data 87 is data about a user from whom the biological measurement data 81 is acquired, and is data expressing a situation of the user. A type of the user feature data 87 is not limited as long as the user feature data 87 is data of a type that has relatively low dependency on a time point and can express a situation of a user. For example, the user feature data 87 can include demographic variables such as age and gender of a user, personality variables measured based on a big five theory, and the like.

Hereinafter, an example will be described in which a set of biological measurement data 81 includes heartbeat interval data, electrodermal activity data, and acceleration data during 30 minutes and a set of subjective value groundtruth data 88 is set to an intensity of the Valence and the Arousal during 30 minutes, unless otherwise specified. The biological measurement data processing device 1 executes processing of training the event evaluation model 90 by using a plurality of sets of the above-described pieces of data.

In data preprocessing S32, the preprocessing unit 52 of the biological measurement data processing device 1 preprocesses data (at least the biological measurement data 81) using the user feature data 87 and the biological measurement data 81. For example, the preprocessing unit 52 executes at least one of processing of correcting individual differences included in the biological measurement data 81, processing of extracting a feature from the biological measurement data 81, processing of reducing the feature extracted from the biological measurement data 81 (a dimension reduction), and the like. For these types of processing, the preprocessing unit 52 may explicitly and sequentially execute a plurality of types of processing as a pipeline, or may execute these types of processing in an end-to-end manner by separately preparing typical processing means.

For example, a series of data preprocessing S32 may be implemented as processing of correcting individual differences, and a dimension reduction of features that is executed by using a principal component analysis (PCA) with the biological measurement data 81 normalized for each user being set as an input, in which data normalization processing based on a series of pieces of the biological measurement data 81 measured from a user in the past is executed and feature extraction processing is not executed.

In the case of processing in an end-to-end manner, processing of removing a measurement noise of the biological measurement data 81 may be set as the data preprocessing S32, and the feature extraction processing and the dimension reduction of features may be implemented in a form of using a latent vector of a variational auto encoder (VAE) which is a deep generative model to which the biological measurement data 81 subjected to noise removal is input.

Normalization processing of a signal scale to be used or noise removal processing may be executed in correcting the individual differences. For example, for each user, for each measurement date, for each measurement date with a user, or for all users, min-max normalization in which normalization is performed with a maximum value and a minimum value, z-score normalization in which normalization is performed with an average and a standard deviation of signals, quantile normalization in which normalization is performed using quantile of a signal intensity distribution, or the like may be used in the normalization processing. When it is known that a signal intensity fluctuates due to aging or the like, deviation value processing or the like of normalizing the signal intensity for each age group may be executed in consideration of age information of a user included in the user feature data 87. Further, clipping processing or winsorize processing in which an abnormal value of a signal is removed to fall within a certain range, movement average processing of preventing and smoothing a sudden fluctuation at a time point, 0th order differential processing using a Savitzky-Golay filter, or the like may be executed in the noise removal processing.

In the processing of extracting a feature from the biological measurement data 81, the feature may be extracted according to a biological signal of the biological measurement data 81 to be used. For example, an average heart rate, a low frequency component (LF) and a high frequency component (HF) that are obtained by a frequency domain analysis and are known to mainly reflect a sympathetic nerve activity or a parasympathetic nerve activity respectively, SDNN, RMSSD, and NN50 that are used in a time domain analysis, a feature using a Lorenz plot used in a nonlinear domain analysis, a feature obtained by a detrended fluctuation analysis, a feature obtained by a complex demodulation method, and the like can be used for heartbeat interval data acquired by the heartbeat sensor 21. A skin conductance level (SCL) or a skin conductance response (SCR) may be used for electrodermal activity data acquired by the electrodermal activity sensor 22. An acceleration norm, the number of times of zero-crossings that is the number of times at which a signal obtained by processing an acceleration norm with a band-pass filter passes through a threshold of ±0.01 G when a gravitational acceleration is set to 1 G, or the like may be used for three-axis acceleration data obtained from the acceleration sensor 23.

Further, predetermined data preprocessing may be executed on the subjective value groundtruth data 88 in addition to the biological measurement data 81. For example, in the case of estimating intensities of the Valence and the Arousal, preprocessing may be executed according to a type of the event evaluation model 90 used in the processing of training the event evaluation model 90 to be described later. For example, in a case where the Valence or the Arousal is measured in five stages from 1 to 5 with a Likert scale, the scale may be converted into a range from −1 to 1 when a regression model is trained. In the case of a classification model, binarization except for 3 which is an intermediate value may be performed, 1 and 2 may be set as a negative example 0, and 4 and 5 may be set as a positive example 1.

A known algorithm can be used in the dimension reduction of features. For example, a principal component analysis, an auto encoder (AE) which is an auto-encoder, uniform manifold approximation and projection (UMAP), and the like may be used in the above-described dimension reduction processing of features.

When typical processing means for preprocessing is separately prepared, a data preprocessing model for data preprocessing may be trained and used as the processing means. In this case, since training processing is typically based on unsupervised learning, the training processing may be performed using the biological measurement data 81 that does not correspond to the subjective value groundtruth data 88. Since the subjective value groundtruth data 88 is acquired at a high cost, with such a configuration, it is possible to train a data preprocessing model from a large amount of the biological measurement data 81, and it is possible to obtain an effect of training a data preprocessing model capable of generating preprocessed data that can be expressed using more various conditions as features.

As described above, the preprocessing unit 52 preprocesses the biological measurement data 81 and preprocesses the subjective value groundtruth data 88 as needed.

In event evaluation model training processing S33, the event score evaluation unit 54 trains the event evaluation model 90 using the preprocessed biological measurement data 81 and the subjective value groundtruth data 88 preprocessed as needed, and generates the event evaluation model 90.

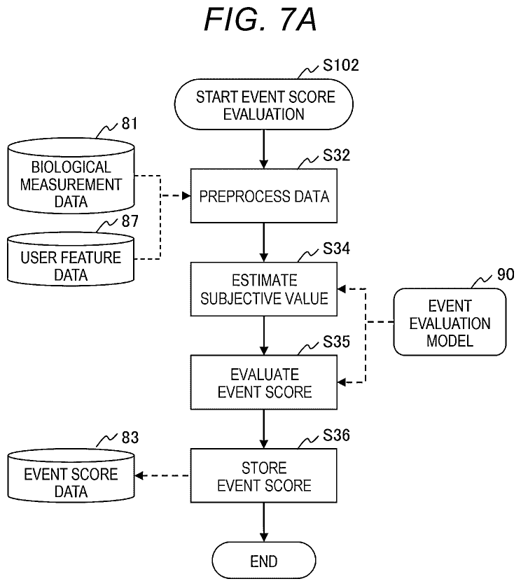

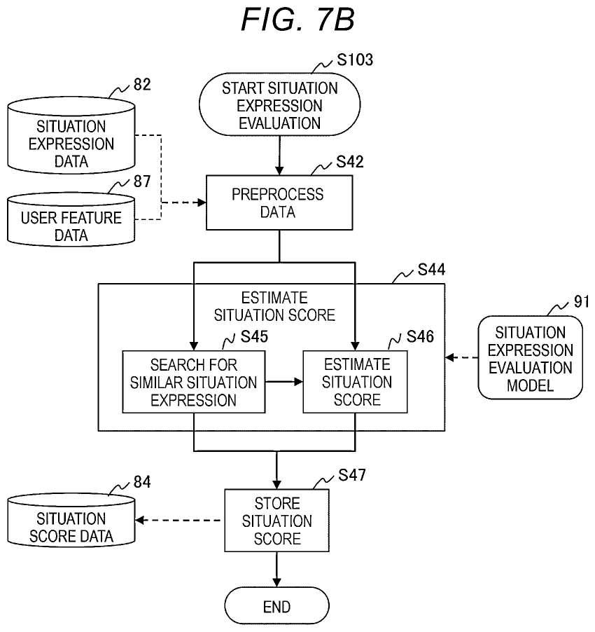

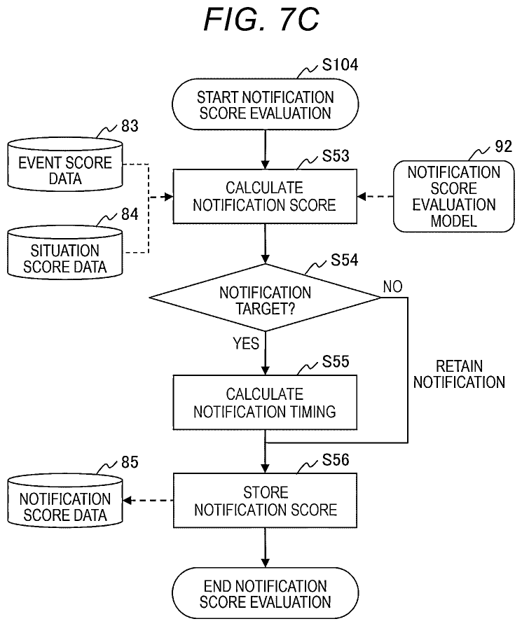

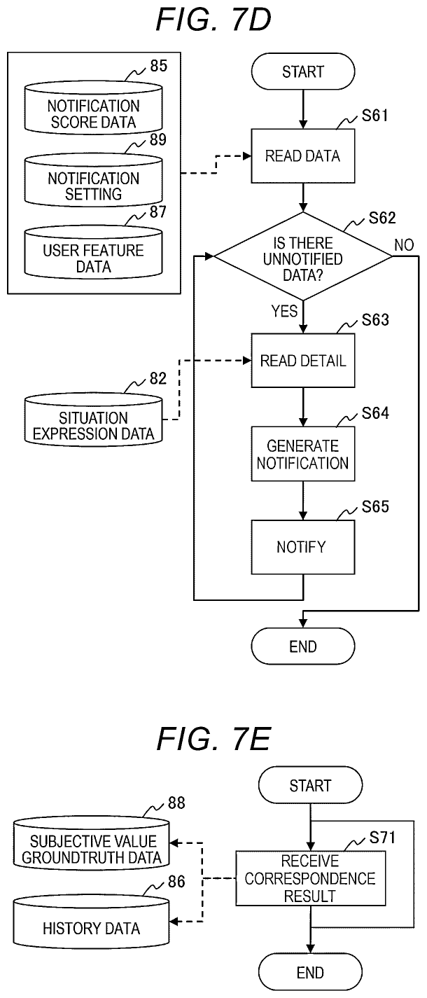

Hereinafter, a case where the event score evaluation unit 54 sequentially executes subjective value estimation processing S34 and event score evaluation processing S35 in event score evaluation processing shown in FIG. 7A will be exemplified. The event score evaluation unit 54 is not limited to a configuration for executing such multi-stage step processing, and may have, for example, a configuration for executing the event score evaluation processing S35 directly based on the biological measurement data 81.

When the event evaluation model 90 for which the subjective value estimation processing S34 is executed by the event score evaluation unit 54 is, for example, a model in which a natural emotion of a user in daily life is an estimation target, the event evaluation model 90 may be trained, by supervised learning, as an emotion dimension estimation model that estimates “intensities of Valence and Arousal during 30 minutes” as the subjective value groundtruth data 88. The subjective value estimation processing S34 can be executed using a known algorithm. For example, a logistic regression model, a decision tree, a random forest, a support vector machine, a neural network, a Bayesian neural network, a deep learning model, and the like can be used as a machine learning algorithm. A classification algorithm and a regression algorithm corresponding to an estimated subjective value can be used as an algorithm. For example, when the intensities of the Valence and the Arousal are estimated by values from −1 to 1, a regression algorithm may be used, and when the intensities of the Valence and the Arousal are estimated by a high or low intensity, a classification algorithm may be used instead of the regression algorithm.

The subjective value groundtruth data 88 as a target of the subjective value estimation processing S34 for the event evaluation model 90 is often data subjectively annotated by a user when the subjective value groundtruth data 88 is particularly obtained in daily life. Therefore, the subjective value groundtruth data 88 may have poor reliability. In consideration of training based on such groundtruth data having poor reliability, the event evaluation model 90 may be implemented by a model suitable for such training. For example, the event evaluation model 90 may be trained using the subjective value groundtruth data 88 weighted according to reliability of a response of each user. In addition, a known algorithm may be used in a model configuration for groundtruth data having poor reliability and a low certainty. For example, although groundtruth data is given to the whole of the biological measurement data 81 for the latest 30 minutes, multiple instance learning may be used based on a fact that it is difficult to strictly determine which time point the groundtruth data corresponds to. When groundtruth data is not determined to be single and reliability of a distribution position or an order of the groundtruth data is relatively high, a Bayesian deep learning model or label distribution learning may be used.

Further, the event evaluation model 90 may be implemented by a plurality of models in consideration of individual differences. In this case, it is possible to estimate a subjective value suitable for each user, and it is possible to obtain an effect of improving suitability to a user and acceptability of a user for a subjective value estimation result.

When the event evaluation model 90 is implemented by a plurality of models in consideration of individual differences, dedicated processing may be added to the event evaluation model 90, or the event evaluation model 90 may be implemented in an end-to-end manner. For example, when dedicated processing is added, data of an estimated subjective value may be obtained by regarding a response style of the subjective value groundtruth data 88 of a user as a recognition tendency of the user, estimating a subjective value applicable to all users using a classification model or a regression model, and then adding correction in consideration of a mid-point response style(MRS) or an extreme response style(ERS) which is a response style to an estimation result of the subjective value. In the case of an end-to-end configuration, the event evaluation model 90 may be implemented by a neural network, may be implemented by multi task learning in which a layer close to a final layer is divided for each user so as to achieve an estimation in which a recognition tendency corresponding to a user is learned, or the event evaluation model 90 may be adapted to a specific user by creating the event evaluation model 90 common to users and finely tuning the event evaluation model 90 for each user.

When training the event evaluation model 90 for which the subjective value estimation processing S34 is executed is completed, in the event score evaluation processing S35, the event score evaluation unit 54 evaluates an event score with the event evaluation model 90 by using an estimated subjective value estimated based on the biological measurement data 81 in the subjective value estimation processing S34. The event score is a value indicating a degree to which confirmation or an intervention behavior is to be performed by a user regarding an event that occurred with the user, and is an index indicating a degree of deviation of the biological measurement data 81 or the estimated subjective value from a usual value.

In the event score evaluation processing S35, the event score evaluation unit 54 uses the event evaluation model 90 to evaluate how much the received biological measurement data 81 or estimated subjective value deviates from a usual value as an event score.

The event evaluation model 90 is typically a model that is trained by unsupervised learning using a known abnormality detection algorithm, and can be configured as a known statistical model or machine learning model. The statistical model may be, for example, a statistical model that outputs a z-score using an average value or a standard deviation in a data distribution of the preprocessed biological measurement data 81 or an estimated subjective value obtained by the subjective value estimation processing S34, a statistical model that outputs a deviation value in the data distribution, a statistical model that outputs quantile in the data distribution, or the like. The machine learning model may be, for example, a machine learning model that outputs a distance with reference to a cluster center of a data distribution estimated non-parametrically based on the preprocessed biological measurement data 81 or an estimated subjective value obtained by the subjective value estimation processing S34.

For example, when an auto encoder which is a typical example of the machine learning model is selected, a sub-model of the event evaluation model 90 used in the event score evaluation processing S35 may be implemented as a model in which either or both of the preprocessed biological measurement data 81 and the estimated subjective value obtained by the subjective value estimation processing S34 are used as an input and a reconstruction error indicating how much the input data can be reconstructed by the auto encoder is calculated. In the case of such a configuration, when the input data is close to the biological measurement data 81 measured at a usual time or an estimated subjective value estimated based on the biological measurement data 81, an event score decreases, and when there is an abnormality in a biological condition or an estimated subjective value or when there is a problem in a measurement condition of a biological condition, an event score increases and thereby a difference from a usual score can be evaluated as an event score.

Although the data preprocessing S32 and the event evaluation model training processing S33 are different from each other in the flowchart shown in FIG. 3, a part of the two types of processing may be executed integrally. For example, the feature extraction processing and the dimension reduction of features described in the data preprocessing S32, the subjective value estimation processing S34, and the event score evaluation processing S35 may be implemented as processing of training the event evaluation model 90 that is executed in an end-to-end manner. In this case, a learning model may be implemented in a form of a deep learning model including a full connection layer and a long short term memory, a graph neural network, a convolutional neural network, self attention, transformer, or the like that is good at handling time-series data.

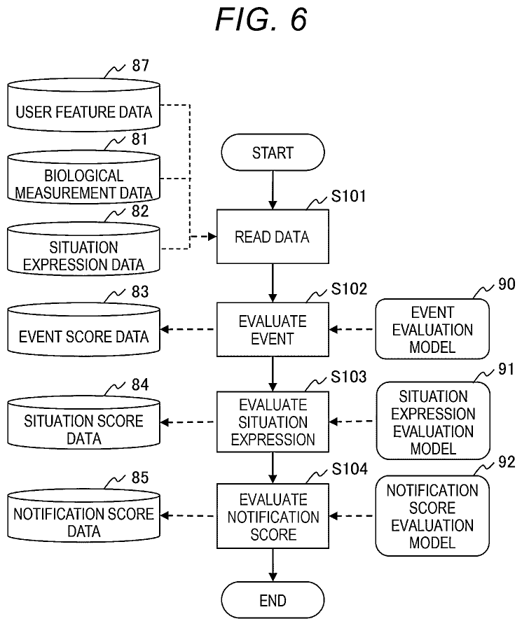

The event evaluation model training processing S33 in the flowchart shown in FIG. 3 is executed at least once before processing of evaluating a degree of an event score shown in FIG. 5 or FIG. 6 to be described later. It is possible to re-train the event evaluation model 90 for the subjective value estimation processing S34 or the event score evaluation processing S35 by executing the processing every predetermined period of time with an increase in the subjective value groundtruth data 88 or the biological measurement data 81. In this manner, a model with higher evaluation accuracy can be generated.

Although a case where the event evaluation model 90 is a multi-stage model used for the subjective value estimation processing S34 and the event score evaluation processing S35 and is trained and used in the processing is described in the present embodiment, typically, it is desirable to prepare a large number of models for processing and use the models together. For example, estimation processing and evaluation processing according to a large number of methods may be prepared for the subjective value estimation processing S34 and the event score evaluation processing S35, so that a large number of estimated subjective values and event scores can be evaluated. In this manner, when an evaluation of a notification score using an event score and a notification determination based on the notification score are performed, which will be described later, the notification determination can be implemented from various viewpoints.

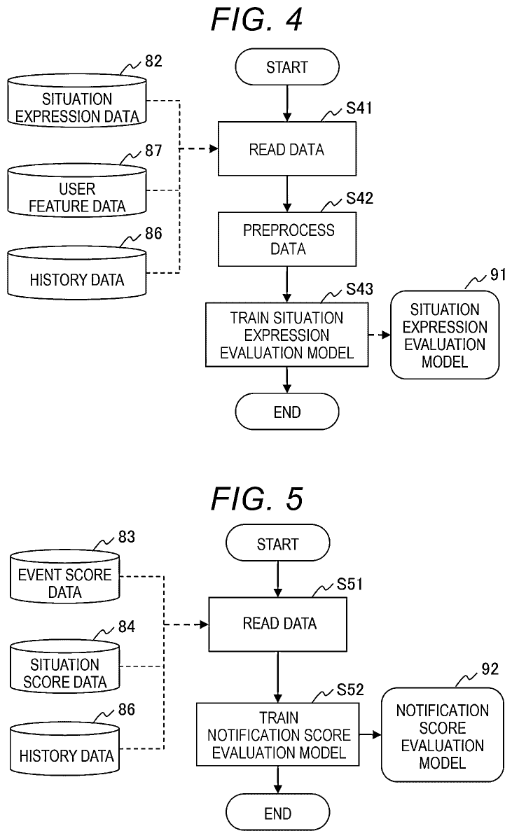

FIG. 4 is a flowchart showing an example of processing of training the situation expression evaluation model 91, which is executed by the biological measurement data processing device 1. The situation expression evaluation model 91 is a model expressing a situation of a user (a situation related to a response to a notification).

In data reading processing S41, the reception unit 51 of the biological measurement data processing device 1 receives the situation expression data 82. The situation expression data 82 is data expressing a situation (a behavior situation) of a user, and is, for example, data reflecting a schedule of a user. The situation expression data 82 includes, for example, a start time point and an end time point of a behavior of a user. The situation expression data 82 may include a situation score for uniquely identifying data. In this case, the reception unit 51 may also receive the user feature data 87 and the history data 86 for a user who receives the situation expression data 82. Hereinafter, a case where the reception unit 51 receives the user feature data 87 and the history data 86 in addition to the situation expression data 82 will be exemplified.

A type of the situation expression data 82 is not limited as long as the situation expression data 82 is data of a type capable of expressing a situation of a user at a certain time point. The situation expression data 82 can include, for example, schedule data of a user including a task of a user and a start time point and an end time point of the task, GPS data that can express a past behavior pattern of a user, and sensor data that can express an activity situation of a user obtained from a sensor such as an acceleration sensor.

The history data 86 comprehensively stores information such as a response to a notification to a user. The history data 86 can include, for example, a start time point and an end time point of a task of a user, a notification situation from a system, and data indicating a response latency until a user responds to a notification. These pieces of data can be acquired as the subjective value groundtruth data 88. The history data 86 is not limited to the subjective value groundtruth data 88, and may include, as data, an item including a response time point indicating a time point when user can respond. In this case, when the situation expression data 82 associated with the history data 86 exists, the situation expression data 82 may be included in the history data 86 in a form in which a correspondence between the situation expression data 82 and the history data 86 can be understood.

In data preprocessing S42, the preprocessing unit 52 executes predetermined preprocessing as needed on the situation expression data 82, the user feature data 87, and the history data 86. For example, when the correspondence between the history data 86 and the situation expression data 82 is clearly defined, a data set obtained by combining these pieces of data may be created, and the history data 86 may be updated.

In situation expression evaluation model training processing S43, the situation score evaluation unit 55 receives the situation expression data 82, the user feature data 87, and the history data 86 that are preprocessed, trains the situation expression evaluation model 91, and generates the situation expression evaluation model 91. The situation score evaluation unit 55 can execute a plurality of types of processing in parallel or sequentially execute a plurality of types of processing in training the situation expression evaluation model 91.

Hereinafter, a case where the situation score evaluation unit 55 executes similar situation expression search processing S45 and situation score estimation processing S46 in situation expression evaluation processing shown in FIG. 7B will be exemplified.

In the similar situation expression search processing S45, the situation score evaluation unit 55 searches for a similar behavior pattern from the situation expression data 82, and estimates a situation (a behavior situation) of a user by using the situation expression evaluation model 91.

In the situation score estimation processing S46, the situation score evaluation unit 55 evaluates a situation score indicating acceptability of a user for a notification by using the situation expression evaluation model 91. The situation score is a value indicating appropriateness of a timing of a notification to a user, and is a value indicating a degree of a situation in which a user easily performs a behavior regarding a notification. Specifically, the situation score is a value in which a situation (a behavior situation) of a user and acceptability of a user for a notification (a possibility that a user can respond to a notification) are associated with each other, and indicates a probability that the user can confirm the notification and can respond to the notification. The situation score can be expressed by, for example, a continuous value from 0 to 1.

In the situation expression evaluation model training processing S43, the situation score evaluation unit 55 that executes the similar situation expression search processing S45 uses the history data 86 updated by being combined with the situation expression data 82 and trains the situation expression evaluation model 91 that searches for a similar behavior pattern from the situation expression data 82. The history data 86 updated by being combined with the situation expression data 82 includes data that can express a behavior pattern such as schedule data or GPS data based on the situation expression data 82.

The situation score evaluation unit 55 can discretize the situation expression data 82 in any predetermined period of time by a predetermined time width and store the discretized situation expression data 82 as a code string of a behavior pattern in the predetermined period of time.

For example, when data that can express a behavior pattern is encoded into a label such as a place or a behavior type, the behavior pattern in a predetermined period of time can be expressed by a code string. In the similar situation expression search processing S45, training is performed by searching for the code string of the behavior pattern using partial match search from the received situation expression data 82. Accordingly, in processing of training the situation expression evaluation model 91 in the similar situation expression search processing S45, a database that stores the code string of the behavior pattern and a partial match search algorithm may be constructed as a sub-model of the situation expression evaluation model 91. A known algorithm related to an array partial match search or a similarity search can be used in the partial match search. The known algorithm includes, for example, a Needleman-Wunsch algorithm and a Smith-Waterman algorithm used for searching for homologous sequences of base sequences.

In the present embodiment, a case where a predetermined period of time is set to one day, the situation expression data 82 is discretized in units of N minutes less than 60 minutes, and the discretized situation expression data 82 is set as a code string of a behavior pattern in the predetermined period of time will be exemplified. In this case, a label of the behavior pattern is encoded as a representative behavior label in units of N minutes. For example, when N is 10 minutes, a behavior pattern in one day is expressed by a code string of 144. In accordance with the code string in this case, the number of times per day that a user can make a response voluntarily and the number of times that the user can respond to a notification are given together with time zone information.

When time granularity of a code string of a behavior pattern is high, an amount of calculation required for the partial match search is enormous, and there is generally a concern that processing is not completed in a calculation time required for use. On the other hand, when a behavior pattern is encoded by a representative behavior label in units of N minutes less than 60 minutes as in the present embodiment, it is possible to obtain an effect that the partial match search can be performed in a realistic calculation time and candidates of a code string of a similar behavior pattern can be extracted.

Since the number of times per day that a user can make a response voluntarily and the number of times that a user can respond to a notification are given to a code string of a behavior pattern together with the time zone information, it is possible to obtain an effect that a situation score indicating acceptability for a notification in a predetermined time zone can be evaluated with a high certainty based on additional information of a code string corresponding to a code string of a searched similar behavior pattern in the situation score estimation processing S46 to be described later.

The situation score evaluation unit 55 that executes the situation score estimation processing S46 trains the situation expression evaluation model 91 based on the situation expression data 82. The situation expression evaluation model 91 is a model expressing a situation related to a response of a user to a notification, and is a model for evaluating a situation score indicating acceptability of a user for a notification in a predetermined time zone. The situation score estimation processing S46 may be implemented according to a type of the situation expression data 82.

For example, in the case of the situation expression data 82 adopting schedule data, the situation score estimation processing S46 can be executed based on the schedule data and the history data 86 in which a response record of a user is recorded.

In this case, for example, when a schedule type is determined in advance, a sub-model of the situation expression evaluation model 91 that estimates a situation score indicating acceptability for a notification corresponding to the schedule type may be constructed based on training based on the schedule type and a response record or domain knowledge. In addition, when the schedule type is not determined in advance, schedule data may be converted into a feature by natural language processing in which a natural sentence related to a schedule type or schedule contents is input, and a sub-model of the situation expression evaluation model 91 that estimates a situation score by supervised learning using a response record for a cluster in which the feature or a similar feature is clustered into a group may be constructed.

Further, when a code string of a corresponding behavior pattern is searched by the similar situation expression search processing S45 described above, the code string of the behavior pattern may be regarded as schedule data having low time granularity, and a sub-model of the situation expression evaluation model 91 may be constructed so as to execute situation score estimation processing for the schedule data.

In addition, for example, in the case of the situation expression data 82 adopting GPS data, a situation score may be estimated based on the GPS data and the history data 86 in which a response record of a user is recorded. In this case, based on temporal information of the GPS data, a model that estimates whether a user is moving or staying, estimates moving means when the user is moving, and estimates a category of a staying place when the user is staying is prepared in advance, and the model can be constructed as a sub-model of the situation expression evaluation model 91. The situation expression evaluation model 91 that estimates a situation score may be constructed by supervised learning of a behavior context such as the obtained moving means or category of the staying place by using, as groundtruth data, prior knowledge related to a response record at that time or acceptability for a notification at the time of a corresponding behavior context.

For example, in the case of the situation expression data 82 adopting sensor data of a sensor (for example, an acceleration sensor or the like) that can express a behavior of a user, a situation score may be estimated based on the sensor data and the history data 86 in which a response record of a user is recorded. In this case, a model that estimates a behavior such as moving means or a life behavior based on the sensor data is prepared in advance, and the model is constructed as a sub-model of the situation expression evaluation model 91. The model can be constructed based on an algorithm or the like known in the field of human activity recognition. The situation expression evaluation model 91 that estimates a situation score may be constructed according to a user behavior estimated by the model or by supervised learning using, as groundtruth data, prior knowledge related to acceptability for a notification at the time of a corresponding behavior.

A series of training processing shown in FIG. 4 is executed at least once before processing of evaluating a degree of a situation score shown in FIG. 5 or FIG. 6 to be described later. It is possible to re-train the situation expression evaluation model 91 for the similar situation expression search processing S45 or the situation score estimation processing S46 by executing the processing every predetermined period of time with an increase in the situation expression data 82 or the history data 86. In this manner, a model with higher evaluation accuracy can be generated.

In the present embodiment, an example in which the situation expression evaluation model 91 is a multi-stage model used in the similar situation expression search processing S45 and the situation score estimation processing S46, and is trained and used for these types of processing is described. Typically, regarding the situation expression evaluation model 91, it is desirable to prepare a large number of models for processing and use these models together. For example, estimation processing and evaluation processing according to a large number of methods may be prepared for the similar situation expression search processing S45 or the situation score estimation processing S46 and examples of the situation score estimation processing S46, so that a large number of situation scores can be evaluated. In this manner, an evaluation of a notification score can be implemented using a situation score, and a notification determination can be implemented from various viewpoints when the notification determination is performed based on the notification score.

FIG. 5 is a flowchart showing an example of processing of training the notification score evaluation model 92, which is executed by the biological measurement data processing device 1. The notification score evaluation model 92 is a model expressing a degree of appropriateness to notify a user, that is, a model expressing a degree to which a timing of a notification is appropriate for a user.

The notification score is a value expressing a degree of appropriateness to notify a user of occurrence of an event, and is an index expressing whether an occurred event is an event of which a user is to be notified. The notification score can be expressed by, for example, a continuous value from 0 to 1.

In data reading processing S51, the reception unit 51 of the biological measurement data processing device 1 receives the event score data 83, the situation score data 84, and the history data 86. The event score data 83 is data including an event score as will be described later. The situation score data 84 is data including a situation score as will be described later. The history data 86 can be used as groundtruth data in training.

In notification score evaluation model training processing S52, the notification score evaluation unit 56 receives the event score data 83, the situation score data 84, and the history data 86, trains the notification score evaluation model 92, and generates the notification score evaluation model 92. The notification score evaluation unit 56 can execute a plurality of types of processing in parallel or sequentially execute a plurality of types of processing in training the notification score evaluation model 92.

Hereinafter, a case where the notification score evaluation unit 56 executes notification score calculation processing S53 and notification timing calculation processing S55 in notification score evaluation processing shown in FIG. 7C will be exemplified.

In the notification score calculation processing S53, the notification score evaluation unit 56 receives the event score data 83, calculates a notification score using the notification score evaluation model 92, and outputs the notification score.

The notification score evaluation unit 56 that executes the notification score calculation processing S53 receives the event score data 83, the situation score data 84, and the history data 86 in the notification score evaluation model training processing S52, and trains the notification score evaluation model 92.

The notification score evaluation unit 56 can obtain a notification score as, for example, a continuous value by performing identity transformation on a value obtained as an event score. In addition, the notification score evaluation unit 56 can calculate a notification score by converting a certainty of an event to be focused on into discrete values using a predetermined step function such as low, medium, and high. The notification score evaluation unit 56 can construct a model that can calculate a notification score in such a manner as the notification score evaluation model 92. When there are a plurality of types of event score data 83, normalization processing may be executed on a domain of each type of event score data 83, and a notification score may be calculated as, for example, a continuous value from 0 to 1. In this manner, a sub-model of the notification score evaluation model 92 that calculates at least an execution time point of notification score calculation processing and a notification score corresponding to the time point can be constructed.

The notification score evaluation unit 56 that executes the notification timing calculation processing S55 evaluates a notification timing based on the execution time point of the notification score calculation processing obtained in the notification score calculation processing S53 and a situation score, and obtains a timing when a user is to be notified of detection of an event.

For example, when immediacy of a notification (occurrence of an event is immediately notified) is required by a user, such as when a certainty of an event related to a subjective health condition of an estimation target is high, it is desirable that a notification timing is immediately as close as possible to the execution time point of the notification score calculation processing S53 regardless of acceptability of the user for the notification. On the other hand, when the immediacy of a notification is less likely to be regarded as important, such as when the certainty is low or when importance of an occurred event is low, it is desirable that a notification timing is a timing when acceptability of a user for a notification is high.

In the notification timing calculation processing S55, the notification score evaluation unit 56 calculates a notification timing candidate with reference to situation scores in a predetermined time range from the execution time point of the notification score calculation processing to a time point when a predetermined time elapses in the situation score data 84. For example, when the predetermined time range is determined to be in one day, a time point at which a value of a situation score indicates a change equal to or larger than a predetermined threshold can be calculated as a notification timing candidate with reference to time-series evaluated situation scores in the day after the execution time point. In this case, a sub-model of the notification score evaluation model 92 can be constructed by optimizing a threshold and the number of notification timing candidates based on a response record in the history data 86.

The notification score evaluation unit 56 obtains a notification score learning model that can evaluate the notification score data 85 by using the event score data 83 and the situation score data 84 as inputs through the above-described processing of training the notification score evaluation model 92. The notification score data 85 is data that includes at least a notification score indicating a certainty of an event to be focused on (a value expressing whether the event is an event of which a user is to be notified) and that can include a notification timing candidate for each acceptability of a notification timing.

In this manner, the notification score evaluation unit 56 can calculate an index for considering immediacy and acceptability for a notification.

A series of training processing shown in FIG. 5 is executed at least once before processing of evaluating a degree of a notification score shown in FIG. 6 to be described later. It is possible to re-train the notification score evaluation model 92 for the notification score calculation processing S53 and the notification timing calculation processing S55 by executing the processing every predetermined period of time with an increase in the event score data 83, the situation score data 84, and the history data 86. In this manner, a model with higher evaluation accuracy can be generated.

FIG. 6 is a flowchart showing an example of processing of evaluating a notification score based on the biological measurement data 81 and the situation expression data 82, which is executed by the biological measurement data processing device 1.

In data reading processing S101, the reception unit 51 of the biological measurement data processing device 1 receives the biological measurement data 81 up to a current time point t and the situation expression data 82 up to the time point t or a time point (t+T). The time point (t+T) is a future time after the time point t by a time T. The reception unit 51 may also receive the user feature data 87 according to a configuration of each trained model.

Here, in view of processing over time, since the biological measurement data 81 is actual data, only data up to a processing time point (the current time point t) can exist. On the other hand, when the situation expression data 82 is, for example, schedule data, data to be scheduled at the time point t and after the time point t which is a target time point can exist. Therefore, in the processing of evaluating a notification score, not only the situation expression data 82 up to the time point t but also the situation expression data 82 up to the time point (t+T) can be used.

In event score evaluation processing S102, the event score evaluation unit 54 receives the event evaluation model 90, generates the event score data 83 based on the biological measurement data 81 and the user feature data 87 using the event evaluation model 90, and outputs the event score data 83. The event score evaluation processing S102 will be described later.

In situation expression evaluation processing S103, the situation score evaluation unit 55 receives the situation expression evaluation model 91, generates the situation score data 84 based on the situation expression data 82 and the user feature data 87 using the situation expression evaluation model 91, and outputs the situation score data 84. The situation expression evaluation processing S103 will be described later.

Although the event score evaluation processing S102 and the situation expression evaluation processing S103 are executed in this order in the present embodiment, the event score evaluation processing S102 and the situation expression evaluation processing S103 may be executed in parallel.

In notification score evaluation processing S104, the notification score evaluation unit 56 receives the notification score evaluation model 92, generates the notification score data 85 based on the event score data 83 and the situation score data 84 by using the notification score evaluation model 92, and outputs the notification score data 85. The notification score evaluation processing S104 will be described later.

As described above, the biological measurement data processing device 1 according to the present embodiment evaluates the notification score data 85 based on the situation score data 84 that is estimated based on the situation expression data 82 and indicates appropriateness of a notification timing, in addition to the event score data 83 that is estimated based on the biological measurement data 81 and indicates a certainty at which a notification is to be issued, and outputs the notification score data 85. Therefore, the biological measurement data processing device 1 according to the present embodiment can obtain a scale for issuing a notification at a timing in consideration of both appropriateness and immediacy when a user is notified of occurrence of a target event.

FIG. 7A is a flowchart showing a detailed example of the event score evaluation processing S102 (FIG. 6) executed by the event score evaluation unit 54 of the biological measurement data processing device 1.

The preprocessing unit 52 of the biological measurement data processing device 1 executes the data preprocessing S32 (FIG. 3) on the biological measurement data 81 and the user feature data 87 that are received up to the time point t.

Thereafter, the event score evaluation unit 54 sequentially executes the subjective value estimation processing S34 and the event score evaluation processing S35 using the received event evaluation model 90, the biological measurement data 81 and the user feature data 87 that are subjected to the data preprocessing, and obtains the event score data 83 up to the time point t.

Thereafter, the event score evaluation unit 54 executes event score storing processing S36 and stores the obtained event score data 83.

FIG. 7B is a flowchart showing a detailed example of the situation expression evaluation processing S103 (FIG. 6) executed by the situation score evaluation unit 55 of the biological measurement data processing device 1.

The preprocessing unit 52 of the biological measurement data processing device executes the data preprocessing S42 (FIG. 4) on the situation expression data 82 and the user feature data 87 that are received up to the time point t or the time point (t+T).

Thereafter, the situation score evaluation unit 55 executes situation score estimation processing S44 including the similar situation expression search processing S45 and the situation score estimation processing S46 using the received situation expression evaluation model 91, and the situation expression data 82 and the user feature data 87 that are subjected to the data preprocessing, and obtains the situation score data 84 up to the time point t or the time point (t+T).

Thereafter, the situation score evaluation unit 55 executes situation score storing processing S47 and stores the obtained situation score data 84 up to the time point t or the time point (t+T).

Although an evaluation value (a situation of a user) obtained in the similar situation expression search processing S45 is also used as an input in the situation score estimation processing S46 in the present embodiment, the evaluation value may not be used in this manner. For example, the evaluation value obtained in the similar situation expression search processing S45 may not be used as an input in the situation score estimation processing S46, and the similar situation expression search processing S45 and the situation score estimation processing S46 may be executed in parallel. In addition, only one of the similar situation expression search processing S45 and the situation score estimation processing S46 may be executed according to the type of the situation expression data 82, and the situation expression evaluation processing S103 may be executed for each piece of the situation expression data 82.

FIG. 7C is a flowchart showing a detailed example of the notification score evaluation processing S104 (FIG. 6) executed by the notification score evaluation unit 56 of the biological measurement data processing device 1.

The notification score evaluation unit 56 executes the notification score calculation processing S53 using the received notification score evaluation model 92, event score data 83, and situation score data 84. The notification score evaluation unit 56 can calculate a notification score up to the time point t based on the event score data 83 up to the time point t and the situation score data 84 up to the time point t. In addition, the notification score evaluation unit 56 can calculate a notification score up to the time point (t+T) based on the event score data 83 up to the time point t and the situation score data 84 up to the time point (t+T). In this manner, the notification score evaluation unit 56 can appropriately obtain a notification timing including a future time after the time point t by the time T.

The notification score evaluation unit 56 executes notification target determination processing S54 on the notification score calculated in the notification score calculation processing S53. In the notification target determination processing S54, for example, in a case where the notification score is expressed by a continuous value from 0 to 1, when a value of the notification score exceeds a predetermined notification score threshold (for example, 0.7), the notification score evaluation unit 56 determines that an event is a notification target, executes the notification timing calculation processing S55, and calculates a notification timing candidate. When the notification score evaluation unit 56 determines that an event is not a notification target, the notification score evaluation unit 56 retains a notification and does not execute the notification timing calculation processing S55.

Thereafter, the notification score evaluation unit 56 executes notification score storing processing S56, and stores the notification score data 85 that can include at least a notification score and a notification timing candidate.

A configuration in which the notification score evaluation unit 56 executes the notification timing calculation processing S55 only when it is determined that an event is a notification target based on a notification score is exemplified in the present embodiment. With such a configuration, a calculation time required for calculating a notification timing can be shortened. Alternatively, the notification score evaluation unit 56 may execute the notification timing calculation processing S55 regardless of whether an event is a notification target.

FIG. 7D is a flowchart showing a detailed example of notification determination processing executed by the notification determination unit 57 of the biological measurement data processing device 1.

The notification determination unit 57 receives the notification score data 85, the notification setting 89, and the user feature data 87 in data reading processing S61. The notification setting 89 is a structured file in which a predetermined threshold (a notification score threshold) for determining whether an event is a notification target and setting for notification generation processing S64 to be described later are stored.

The notification determination unit 57 executes processing S62 of determining whether there is unprocessed data in the notification score data 85. When there is unprocessed data, subsequent processing S63 to processing S65 related to a notification determination are executed until there is no unprocessed data. In the following description, a case where the subsequent processing S63 to processing S65 related to a notification determination are executed in parallel for each user and are executed sequentially from an earliest notification timing candidate will be exemplified.

In the processing S63 and processing S64, the notification determination unit 57 determines whether to notify a user of an event (notification determination processing).

In the details reading processing S63, the notification determination unit 57 receives the situation expression data 82 corresponding to a situation score ID using the situation score ID stored in the notification score data 85 as a key.

In the details reading processing S63, among a plurality of notification scores calculated at a plurality of time points, the notification determination unit 57 collectively handles notification scores whose calculation time points are within a predetermined time range as a group of notification scores, and executes the notification determination processing based on the group of notification scores, whereby a user can be notified of a plurality of notifications at close time points (a plurality of notifications are within a predetermined time range) in a collective manner.

For example, regarding the notification score data 85 being processed, when there are a plurality of pieces of the notification score data 85 for the same user at close time points for the biological measurement data 81 for which at least one of a start time point and an end time point is repeated, the notification determination unit 57 receives the situation expression data 82 corresponding to all pieces of the notification score data 85. In this manner, when there are a plurality of notifications at close time points, the plurality of notifications can be collectively received and a user can be notified of the plurality of notifications in a collective manner.

Thereafter, the notification determination unit 57 executes the notification generation processing S64 based on the target notification score data 85, the target situation expression data 82, the notification setting 89, and the user feature data 87. In the notification generation processing S64, the notification determination unit 57 determines whether an event is an event of which a user is to be notified based on a notification score and a notification timing candidate stored in the notification score data 85, a notification condition set in the notification setting 89, and notification setting for each user (setting for each user related to a notification) to be described later in the user feature data 87, and when the event is a notification target, the notification determination unit 57 generates notification contents.

As described above, in the notification generation processing S64, the notification determination unit 57 generates notification contents when an event is a notification target (when the event is an event of which a user is to be notified) based on a notification score, a notification timing candidate, a notification condition, and notification setting for each user. For example, for a plurality of notification conditions, when a notification score exceeds a notification score threshold and one of notification timing candidates is close to a time point of a processing time point and exceeds a notification timing threshold, the notification determination unit 57 generates notification contents based on the notification score data 85. The notification timing threshold is a value indicating acceptability of a user for a notification, and whether to notify a user can be determined according to the value.

The notification contents may include, for example, a notification indicating that a strong emotion occurs when occurrence of a strong emotion is set as a target event and a notification requesting a response indicating what emotion a user has in what situation. When occurrence of strong stress or an unstable condition is set as a target event, the notification contents may include a situation notification, a proposal of an intervention measure for improving a situation, and a notification requesting a response about whether a user actually performs a behavior to improve a situation.

The notification output unit 58 executes the notification processing S65, and outputs a notification indicating occurrence of an event by outputting the generated notification contents.

When the notification determination unit 57 collectively handles a plurality of notification scores as a group of notification scores and executes the notification determination processing based on the group of notification scores, the notification determination unit 57 executes the notification generation processing S64 based on a group of target notification score data 85, a group of target situation expression data 82, the notification setting 89, and the user feature data 87. The notification output unit 58 executes the notification processing S65 and collectively outputs a plurality of notifications indicating occurrence of an event to a user at one time by collectively outputting a plurality of the generated notification contents.

Since a plurality of pieces of notification score data 85 for the same user at close time points where a start time point and an end time point of the notification score data 85 overlap with each other are collectively handled as a group of notification score data 85 and the user is notified of the group of notification score data 85 in a collective manner, when a degree of a notification score or a notification timing candidate is changed together with the elapse of time, notification contents can be generated in consideration of appropriateness and immediacy in view of the latest situation, and further, it is possible to prevent issue of a plurality of notifications regarding similar events. As a result, it is possible to reduce annoyance that similar notifications are issued a plurality of times for a user, and it is possible to obtain an effect of improving appropriateness and acceptability for each notification.

Based on predetermined setting, for example, a length of a notification (a notification length) set in the notification setting 89, the notification output unit 58 can determine at least one of an amount of information to be presented (for example, a length of a message) and an amount of a response required for a user (for example, the number of items input by a user) in a notification to be output. Accordingly, the biological measurement data processing device 1 according to the present embodiment can send, to the user terminal 7, a notification of an amount and contents appropriate for a user.

For example, the notification output unit 58 may adjust a notification or an amount of a response required for a user by a notification based on a length of a notification set in the notification setting 89, and generate notification contents based on the situation expression data 82 (including a group of situation expression data 82). Accordingly, even when a value of a notification score is slightly low, it is possible to generate the notification contents according to needs of a user or an administrator who wants to know or respond to a detected event in a short time, and it is possible to obtain an effect that a notification can be issued in an appropriate amount in view of a notification score while ensuring immediacy of the notification.

In the notification processing S65, the input and output device 5 executes notification processing on a notification target via the network 9 based on the generated notification contents. For example, when the notification target is a user, the input and output device 5 executes the notification processing S65 of notifying the user terminal 7 of the user, and the notification device 15 (FIG. 1) of the user terminal 7 issues a notification to the user. At this time, when the user terminal 7 is a smartphone or a smartwatch, a notification can be issued in a form of a push notification to the smartphone or the smartwatch, a message to a notification application for a user, and the like. When the notification target is an administrator, the input and output device 5 executes the notification processing S65 of notifying the administrator terminal 8 of the administrator who collects a user corresponding to a user ID of determination result data, and the notification device 33 (FIG. 1) of the administrator terminal 8 issues a notification to the administrator. At this time, when the administrator terminal 8 is a personal computer (PC), a notification can be issued in a form of an output of a warning sound to the PC, a notification to the PC by an E-mail, display of a notification to an administrator application, and the like. A notification situation flag is updated to “completed” for the notification score data 85 for which the notification processing is completed.

An example in which an evaluation is performed by setting a plurality of notification score thresholds and a plurality of notification timing thresholds for a user is described in the present embodiment. In addition, a notification score threshold and a notification timing threshold may be separately set for an administrator in addition to the user. In this case, a notification determination can be made by a user and an administrator with different criteria according to a notification target, and a situation of which a user or an administrator is to be notified can be determined according to a behavior to be taken by a notification receiver.

As described above, when a user is notified of occurrence of a target event using the notification score, it is possible to issue a notification at a timing in consideration of appropriateness and immediacy, and it is possible to obtain an effect of promoting a behavior of a user based on the notification, such as improvement of acceptability of an intervention measure based on the notification.

FIG. 7E is a flowchart showing an example of processing of receiving a correspondence result according to a notification output, which is executed by the biological measurement data processing device 1.

The reception unit 51 of the biological measurement data processing device 1 executes correspondence result receiving processing S71 for receiving a response from a target terminal on which the notification processing is executed. Hereinafter, a case where a user is notified at a timing when the user is assumed to be in a subjective condition of an emotion different from a usual emotion based on a notification score will be described as an example of receiving a response from the user terminal 7. A user uses the user terminal 7 to respond to a notification (inputs subjective value groundtruth data to the user terminal 7). In this case, in the correspondence result receiving processing S71, the reception unit 51 receives, as the subjective value groundtruth data 88, groundtruth data expressing a subjective condition of an emotion generated by the user terminal 7 based on a response of a user. When the reception unit 51 receives the subjective value groundtruth data 88, the reception unit 51 updates a correspondence situation flag of the history data 86 to “completed”.

As described above, the biological measurement data processing device 1 can obtain a result of a behavior performed by a user who receives a notification for the notification issued at a timing appropriate for an intervention measure or data collection, such as a timing when the user is in a subjective condition different from a usual condition. For example, in the present embodiment, when the subjective value groundtruth data 88 is acquired as groundtruth data of an emotion corresponding to a moment in response to a notification related to a timing when a user is assumed to be in a subjective condition of an emotion different from a usual emotion, it is possible to efficiently acquire groundtruth data related to an emotion that occurs rarely in daily life. When a flag value indicating whether a notification is confirmed by a user, contents that are handled, and an actual subjective health condition at that time are acquired for a notification related to a timing when a user is assumed to be in a subjective health condition such as fatigue or a mood different from a usual condition, the biological measurement data processing device 1 can acquire correspondence result data to be used for verifying subjective value estimation accuracy or evaluating an intervention effect.

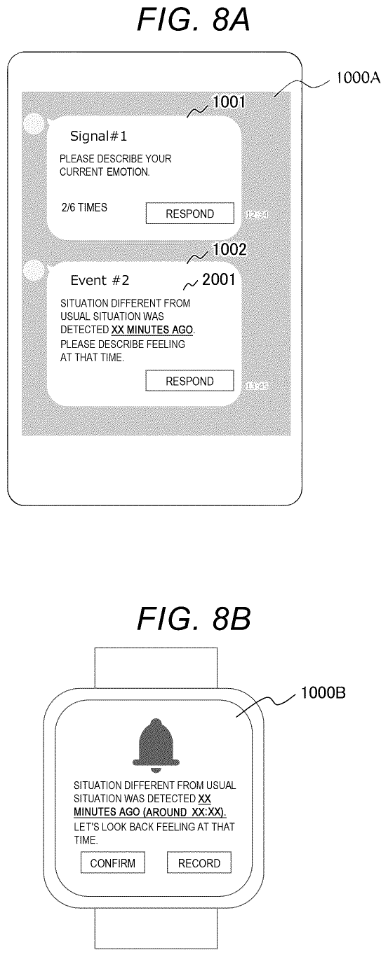

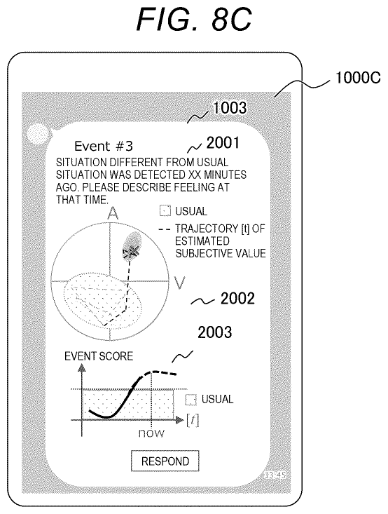

FIGS. 8A, 8B, and 8C are diagrams showing examples of notification output screens displayed on the screen of the user terminal 7 by the result display unit 59. An example in which a notification is displayed in a form of a message to a notification application for a user will be described in the present embodiment.

A screen 1000A shown in FIG. 8A is an example of a display screen based on a notification score. FIG. 8A shows a case where the user terminal 7 is a smartphone.

In a device in the related art, for example, a notification application displays a notification 1001 to a user at an irregular interval, such as Signal #1 shown in FIG. 8A, and attempts to collect groundtruth data of a corresponding emotion at a certain moment. However, since an emotion such as extreme happiness or sadness does not frequently occur in daily life, a long period of time may be taken to collect groundtruth data with a notification issued simply at an irregular interval, or a predetermined period of time may end without acquiring sufficient groundtruth data. It cannot be denied that a scene in which a strong emotion occurs is a scene different from usual life, and even when occurrence of a strong emotion is detected while the scene different from usual life continues and a user is immediately notified of this, an effective response to the notification cannot be obtained in many cases.

When a notification score according to the present embodiment is used, a timing when a user is in a subjective condition (an emotion condition) different from a usual condition can be displayed on the user terminal 7 as a notification 1002 in a format such as Event #2 in consideration of appropriateness of a notification indicating whether the notification at a time point of a calculated notification timing candidate is easily accepted by a user, in addition to immediacy of a notification at a time point when an event such as a strong emotion occurs. In the notification 1002, instead of an immediate notification, information related to a time when a detected event occurs can be expressed based on a notification score, for example, as in a message 2001. Accordingly, it is possible to obtain an effect that a user can obtain a material for introspecting notification contents when the subjective value groundtruth data 88 related to a detected event and the history data 86 are generated, even when a notification is not immediate.

In this manner, compared with the device in the related art that acquires the subjective value groundtruth data 88 which is groundtruth data of an emotion based on an irregular notification such as the Signal #1 in the notification 1001, it is possible to obtain an effect in the present embodiment that a response of a user to a notification is received as the subjective value groundtruth data 88 and the history data 86 in the correspondence result receiving processing, and thus it is possible to acquire the subjective value groundtruth data 88 corresponding to a rare emotion condition with high efficiency.

A screen 1000B in FIG. 8B is another example of the display screen based on the notification score. FIG. 8B shows a case where the user terminal 7 is a smartwatch. Display contents of the screen 1000B is not substantially different from display contents of the screen 1000A, and instead of an immediate notification, information related to a time when a detected event occurs is displayed based on a notification score. When the user terminal 7 is a smartwatch, it is possible to obtain an effect that a user can be effectively notified using other notification means such as a vibration in addition to display, as compared with a smartphone.

A screen 1000C shown in FIG. 8C is an example of a display screen based on a notification score and an event score. In a notification 1003 in a format of Event #3 on the screen 1000C, a timing when a user is in a subjective condition different from a usual condition can be displayed on the user terminal 7 as in the message 2001 based on a notification score in a similar manner to that shown on the screen 1000A.

Further, on the screen 1000C, transition of an event score serving as a basis of calculation of a notification score is drawn as, for example, display 2002 or display 2003, and a user can be notified of the transition.

The display 2002 is an example in which a temporal change of an estimated subjective value included in the event score data 83 is displayed on an estimated subjective value plane with the intensity of the Valence being set as an x axis and the intensity of the Arousal being set as a y axis. In the display 2002, the higher a density of dotted lines is, the more recent a notification condition is. In the display 2002, a usual condition of a user is indicated by shading, and it is possible to clearly notify that the latest estimated subjective value remarkably deviates from a usual condition.

The display 2003 is an example showing a temporal change in an event score of the event score data 83. Similar to the display 2002, a range of event scores indicating that a user is in a usual condition is indicated by shading in the display 2003. As compared with the display 2002 which is an example in which information is shown on the estimated subjective value plane, the display 2003 can easily present to a user regarding whether a situation is different from a usual situation using one index such as an event score. In this manner, by notifying a user of the event score in addition to the notification score, the user can be notified of whether a notification is an immediate notification, and can obtain information for understanding why and how a situation is different from a usual situation, thereby obtaining an effect of improving accuracy of introspection of notification contents.

As described above, the result display unit 59 can display at least one of a display screen based on a notification score and a display screen based on an event score on the screen of the user terminal 7.

Next, a feature structure of data used in the biological measurement data processing device 1 will be described.

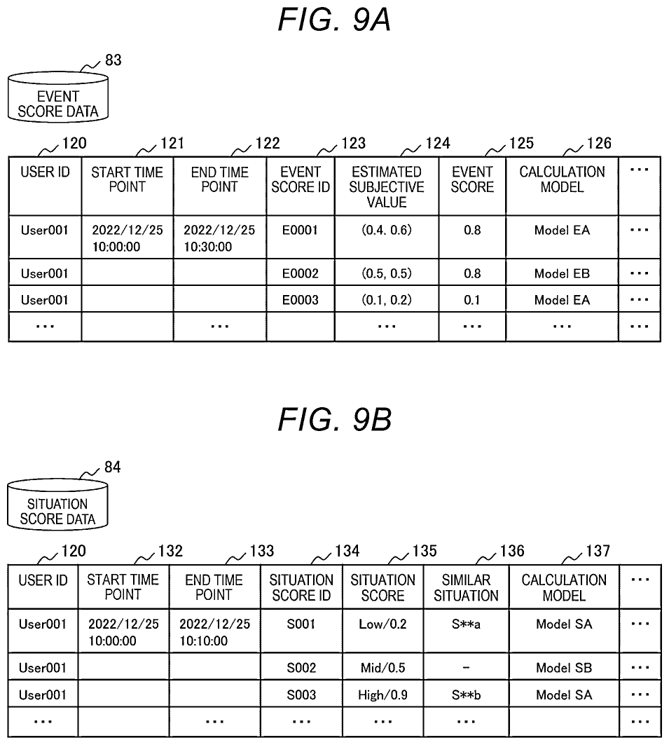

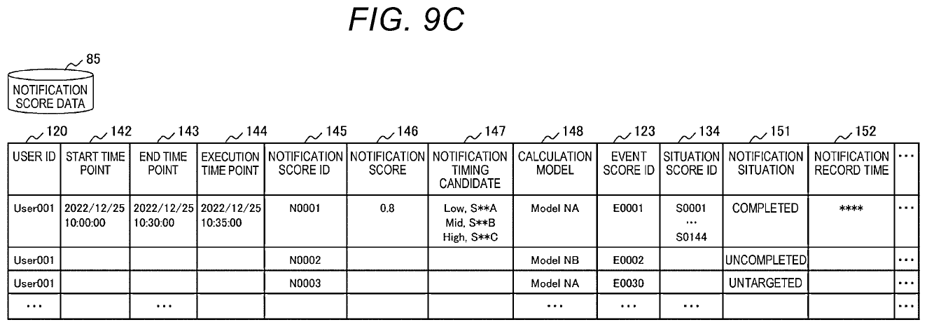

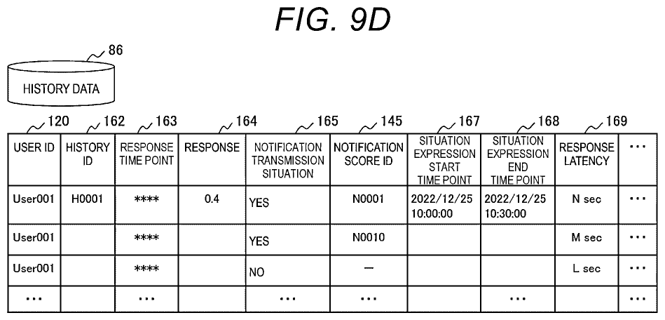

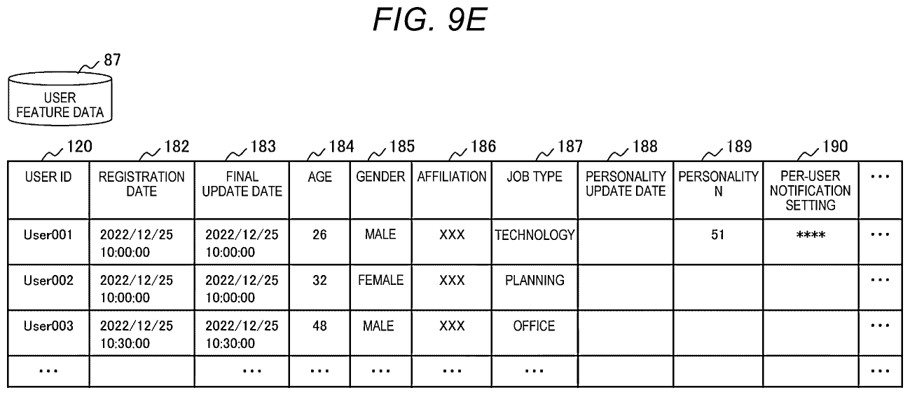

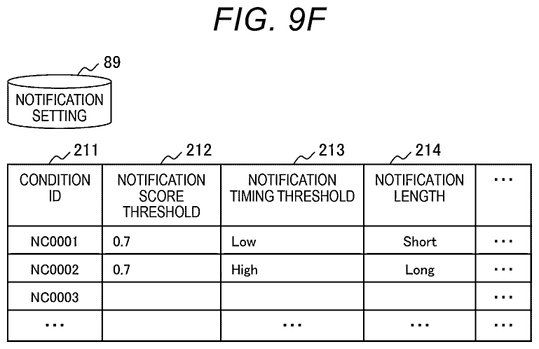

FIG. 9A is a diagram showing an example of a data structure of the event score data 83 stored by the biological measurement data processing device 1. Typically, the event score data 83 stores a user ID 120, a start time point 121 and an end time point 122 of an event, an event score ID 123 for uniquely specifying data of the event score data 83, an estimated subjective value 124, and an event score 125. In the example shown in FIG. 9A, the estimated subjective value 124 is shown as a set of the intensity of the Valence and the intensity of the Arousal as shown in the display 2002 in FIG. 8C. In addition, when there are a plurality of types of event evaluation models 90, the event score data 83 may store a calculation model 126 which is information for specifying the used event evaluation model 90.

FIG. 9B is a diagram showing an example of a data structure of the situation score data 84 stored by the biological measurement data processing device 1. Typically, the situation score data 84 stores the user ID 120, a start time point 132 and an end time point 133 of a situation (a behavior) of a user, a situation score ID 134 for uniquely specifying data of the situation score data 84, and a situation score 135. When the similar situation expression search processing S45 is executed, the situation score data 84 may store a similar situation 136. The similar situation 136 is information for uniquely specifying a behavior pattern when searching for a similar behavior pattern in the similar situation expression search processing S45. A past similar situation (behavior pattern) can be searched for using the similar situation 136. In addition, when there are a plurality of types of situation expression evaluation models 91, the situation score data 84 may store a calculation model 137 which is information for specifying the used situation expression evaluation model 91.

FIG. 9C is a diagram showing an example of a data structure of the notification score data 85 stored by the biological measurement data processing device 1. Typically, the notification score data 85 stores the user ID 120, a start time point 142 and an end time point 143 of an event, an execution time point 144 of a notification score evaluation, a notification score ID 145 for uniquely specifying data of the notification score data 85, a notification score 146, and a notification timing candidate 147. When there are a plurality of types of notification score evaluation models 92, the notification score data 85 may store a calculation model 148 which is information for specifying a used model. Further, in order to associate an event score and a situation score information used for calculating a notification score with each other, the notification score data 85 may store the event score ID 123 and the situation score ID 134. In addition, a notification situation 151, a notification record time 152, and the like may be stored in order to record a notification situation. The notification situation 151 is information indicating whether a notification is actually issued. The notification record time 152 is information indicating a time point when a notification is actually issued.

FIG. 9D is a diagram showing an example of a data structure of the history data 86 stored by the biological measurement data processing device 1. Typically, the history data 86 stores the user ID 120, a history ID 162 for uniquely specifying data of the history data 86, a response time point 163, and a response 164. The response 164 is any value input by a user other than a subjective value. When information about a response to a notification is supplemented, the history data 86 may store a notification transmission situation 165, the notification score ID 145, a situation expression start time point 167, a situation expression end time point 168, and a response latency 169 that is a time required from a time when a notification is issued to a time when a response is given. The situation expression start time point 167 and the situation expression end time point 168 respectively correspond to the start time point 142 and the end time point 143 of the notification score data 85.

FIG. 9E is a diagram showing an example of a data structure of the user feature data 87 stored by the biological measurement data processing device 1. Typically, the user feature data 87 stores the user ID 120, a registration date 182, a final update date 183, age 184, and gender 185. The user feature data 87 may store, for example, an affiliation 186 and a job type 187 as wider demographic variables.

Further, when personality temperament information is used as a feature, the user feature data 87 may additionally store information such as personality update date 188 which is a date on which a personality variable is measured based on a big five theory, and personality N 189 which indicates neuroticism among personality variables. In addition, when setting per user related to a notification is approved, the user feature data 87 may store per-user notification setting 190.

FIG. 9F is a diagram showing an example of a data structure of the notification setting 89 stored by the biological measurement data processing device 1. Typically, the notification setting 89 stores a condition ID 211 for specifying a notification condition, and notification condition details. The notification condition details include, for example, a notification score threshold 212, a notification timing threshold 213, and a notification length 214.

Although FIGS. 9A to 9F show examples in which parameters of data are assumed to be fixed amounts and the data is expressed in table formats, data to be used by the biological measurement data processing device 1 may not be expressed in the formats shown in FIGS. 9A to 9F. For example, when the notification condition details are changed according to a type of a notification condition, data of the notification setting 89 may be stored in a simple structured data format.

As described above, the biological measurement data processing device 1 according to the present embodiment includes the reception unit 51 configured to receive the biological measurement data 81 of a user and the situation expression data 82 expressing a situation of the user, the event score evaluation unit 54 configured to evaluate, based on the biological measurement data 81, an event score indicating a degree to which confirmation or an intervention behavior is to be performed by the user regarding an event that occurred with the user, the situation score evaluation unit 55 configured to evaluate, based on the situation expression data 82, a situation score indicating a degree of a situation in which the user easily performs a behavior regarding a notification, and the notification score evaluation unit 56 configured to calculate, based on the event score and the situation score, a notification score expressing a degree of appropriateness to notify the user of occurrence of the event, and output the notification score.

Accordingly, the biological measurement data processing device 1 according to the present embodiment evaluates a notification score based on a situation score indicating appropriateness of a notification timing estimated based on the situation expression data 82, in addition to the event score 125 indicating a certainty at which a notification is to be issued and that is estimated based on the biological measurement data 81, and outputs the notification score, so that it is possible to issue a notification at a timing in consideration of appropriateness and immediacy by using the notification score when a user is notified of occurrence of an event. According to this effect, it is also possible to obtain a secondary effect of promoting a behavior of a user based on the notification, such as improvement of acceptability for an intervention measure based on the notification.

The invention is not limited to the embodiment described above, and may include various modifications. For example, the embodiment has been described in detail to facilitate understanding of the invention, and the invention is not necessarily limited to those including all configurations described above. A part of configurations of one embodiment can be replaced with a configuration of another embodiment. A configuration of another embodiment can also be added to a configuration of one embodiment. A part of a configuration in each embodiment may be deleted, or may be added with or replaced with another configuration.

A part or all configurations of the device according to the invention may be implemented by hardware, for example, by designing an integrated circuit. A part or all of configurations may be implemented by software such that the processor 2 interprets and executes a program for implementing a function. Information such as a program, a table, a file, measurement information, and calculation information for implementing a function can be recorded in a recording device such as the memory 3, a hard disk drive, and a solid state drive (SSD), or a recording medium such as an IC card, an SD card, and a DVD. Therefore, a component of the device according to the invention can implement a function as a processor, a processing unit, a program module, and the like.

In the drawings, control lines and information lines considered to be necessary for explanation are shown, and not all control lines and information lines necessary for a product are necessarily shown. In an actual product, almost all components may be considered to be connected to one another.

What is claimed is:

1. A biological measurement data processing device comprising:
a reception unit configured to receive biological measurement data of a user and situation expression data expressing a situation of the user;
an event score evaluation unit configured to evaluate, based on the biological measurement data, an event score indicating a degree to which confirmation or an intervention behavior is to be performed by the user regarding an event that occurred with the user;
a situation score evaluation unit configured to evaluate, based on the situation expression data, a situation score indicating a degree of a situation in which the user easily performs a behavior regarding a notification; and
a notification score evaluation unit configured to calculate, based on the event score and the situation score, a notification score expressing a degree of appropriateness to notify the user of occurrence of the event, and output the notification score.

2. The biological measurement data processing device according to claim 1, further comprising:
a notification determination unit configured to perform, based on the notification score, a notification determination of determining whether to notify the user of the event.

3. The biological measurement data processing device according to claim 2, further comprising:
a notification output unit configured to output a notification indicating occurrence of the event based on a result of the notification determination performed by the notification determination unit.

4. The biological measurement data processing device according to claim 3, further comprising:
a result display unit configured to display, on a terminal used by the user, at least one of the notification score calculated by the notification score evaluation unit and the event score evaluated by the event score evaluation unit as the notification indicating occurrence of the event.

5. The biological measurement data processing device according to claim 1, wherein
the event score evaluation unit evaluates the event score up to a time point t,
the situation score evaluation unit evaluates the situation score up to a time point (t+T) that is a time point after the time point t by a time T, and
the notification score evaluation unit calculates the notification score up to the time point (t+T) based on the event score up to the time point t and the situation score up to the time point (t+T).

6. The biological measurement data processing device according to claim 3, wherein
among a plurality of the notification scores calculated at a plurality of time points, the notification determination unit sets the notification scores whose calculation time points are within a predetermined time range as a group of notification scores, and performs the notification determination based on the group of notification scores, and
the notification output unit collectively outputs a plurality of notifications indicating occurrence of the event based on a result of the notification determination performed by the notification determination unit.

7. The biological measurement data processing device according to claim 1, wherein
the situation score evaluation unit discretizes the situation expression data in any period of time by a predetermined time width, and stores the discretized situation expression data as a code string of a behavior pattern in the period of time.

8. The biological measurement data processing device according to claim 3, wherein
the notification output unit determines at least one of an information amount to be presented and a response amount required for the user in the notification output based on predetermined setting.

9. The biological measurement data processing device according to claim 1, wherein
the event that occurred with the user is a change or an abnormality in a subjective condition of the user, and
the subjective condition is a physical and mental condition felt by the user.

10. A biological measurement data processing method comprising:
a reception step of receiving biological measurement data of a user and situation expression data expressing a situation of the user;
an event score evaluation step of evaluating, based on the biological measurement data, an event score indicating a degree to which confirmation or an intervention behavior is to be performed by the user regarding an event that occurred with the user;
a situation score evaluation step of evaluating, based on the situation expression data, a situation score indicating a degree of a situation in which the user easily performs a behavior regarding a notification; and
a notification score evaluation step of calculating, based on the event score and the situation score, a notification score indicating a degree of appropriateness to notify the user of occurrence of the event, and outputting the notification score.

11. The biological measurement data processing method according to claim 10, further comprising:
a notification determination step of performing, based on the notification score, a notification determination of determining whether to notify the user of the event.

12. The biological measurement data processing method according to claim 11, further comprising:
a notification output step of outputting a notification indicating occurrence of the event based on a result of the notification determination in the notification determination step.

13. The biological measurement data processing method according to claim 12, further comprising:
a result display step of displaying, on a terminal used by the user, at least one of the notification score calculated in the notification score evaluation step and the event score evaluated in the event score evaluation step as the notification indicating occurrence of the event.

14. The biological measurement data processing method according to claim 10, wherein
the event that occurred with the user is a change or an abnormality in a subjective condition of the user, and
the subjective condition is a physical and mental condition felt by the user.
